SEQUENCE LISTING (20 sequences)

SEQ ID NO: 1 - DNA, 69 bp, synthetic construct
  source 1..69: mol_type = other DNA
tggatggtagctcggtcggggtgggtgggttggcaagtcttttttttttttttttagtgaggttcgtgt

SEQ ID NO: 2 - DNA, 77 bp, synthetic construct
  source 1..77: mol_type = other DNA
ctgtatccctgtgtccctcactttttttttttttttttggatggtagctcggtcggggtgggtgggttggcaagtct

SEQ ID NO: 3 - DNA, 89 bp, synthetic construct
  source 1..89: mol_type = other DNA
acattgctgcgtgatcgcctcacatgggtttgtctggtcgatttggaggtggtgggtggctttttttttttttttctcatgcttcctcg

SEQ ID NO: 4 - DNA, 97 bp, synthetic construct
  source 1..97: mol_type = other DNA
cttcgtctacatcgtgcatgagtttttttttttttttacattgctgcgtgatcgcctcacatgggtttgtctggtcgatttggaggtggtgggtggc

SEQ ID NO: 5 - DNA, 54 bp, synthetic construct
  source 1..54: mol_type = other DNA
tttttacacgaacacagggatacagccgtgacaagtagctctgtatccctgtgt

SEQ ID NO: 6 - DNA, 54 bp, synthetic construct
  source 1..54: mol_type = other DNA
tttttggatacagagctacttgtcacggctgtatccctgtgtgacaagtagctc

SEQ ID NO: 7 - DNA, 54 bp, synthetic construct
  source 1..54: mol_type = other DNA
tttttcgaggacgatgtagacgaagcccgacgttacttatcttcgtctacatcg

SEQ ID NO: 8 - DNA, 54 bp, synthetic construct
  source 1..54: mol_type = other DNA
tttttagacgaagataagtaacgtcgggcttcgtctacatcgacgttacttatc

SEQ ID NO: 9 - DNA, 69 bp, synthetic construct
  source 1..69: mol_type = other DNA
tggatggtagctcggtcggggtgggtgggttggcaagtctttttttttttttttttcagaccttgctct

SEQ ID NO: 10 - DNA, 77 bp, synthetic construct
  source 1..77: mol_type = other DNA
gtctatgggtctctgggtctgattttttttttttttttggatggtagctcggtcggggtgggtgggttggcaagtct

SEQ ID NO: 11 - DNA, 89 bp, synthetic construct
  source 1..89: mol_type = other DNA
acattgctgcgtgatcgcctcacatgggtttgtctggtcgatttggaggtggtgggtggctttttttttttttttgagatcgttggtgc

SEQ ID NO: 12 - DNA, 97 bp, synthetic construct
  source 1..97: mol_type = other DNA
gttgctgtagatgctcgatctctttttttttttttttacattgctgcgtgatcgcctcacatgggtttgtctggtcgatttggaggtggtgggtggc

SEQ ID NO: 13 - DNA, 54 bp, synthetic construct
  source 1..54: mol_type = other DNA
tttttagagcaagagacccatagacggctcagaactacgtgtctatgggtctct

SEQ ID NO: 14 - DNA, 54 bp, synthetic construct
  source 1..54: mol_type = other DNA
tttttccatagacacgtagttctgagccgtctatgggtctctcagaactacgtg

SEQ ID NO: 15 - DNA, 54 bp, synthetic construct
  source 1..54: mol_type = other DNA
tttttgcaccagcatctacagcaacgggcagcttagttatgttgctgtagatgc

SEQ ID NO: 16 - DNA, 54 bp, synthetic construct
  source 1..54: mol_type = other DNA
tttttacagcaacataactaagctgcccgttgctgtagatgcagcttagttatg

SEQ ID NO: 17 - DNA, 26 bp, synthetic construct
  source 1..26: mol_type = other DNA
aaaaaaaaaaaaagtgaggttcgtgt

SEQ ID NO: 18 - DNA, 34 bp, synthetic construct
  source 1..34: mol_type = other DNA
ctgtatccctgtgtccctcacttttttttttttt

SEQ ID NO: 19 - DNA, 26 bp, synthetic construct
  source 1..26: mol_type = other DNA
aaaaaaaaaaaactcatgcttcctcg

SEQ ID NO: 20 - DNA, 34 bp, synthetic construct
  source 1..34: mol_type = other DNA
cttcgtctacatcgtgcatgagtttttttttttt

(19) United States
(12) Patent Application Publication
(10) Pub. No.: US 2025/0314659 A1
(43) Pub. Date: Oct. 9, 2025

(54) IN-SITU MEMBRANE PROTEIN DIMERIZATION STATE DETECTION KIT AND USE THEREOF

(71) Applicant: Nanjing University of Posts and Telecommunications, Jiangsu (CN)

(72) Inventors: Chunyuan SONG, Jiangsu (CN); Lianhui WANG, Jiangsu (CN)

(73) Assignee: Nanjing University of Posts and Telecommunications, Jiangsu (CN)

(21) Appl. No.: 18/865,685

(22) PCT Filed: Oct. 14, 2022
(86) PCT No.: PCT/CN2022/125363

§ 371 Date: Nov. 14, 2024

(30) Foreign Application Priority Data
May 16, 2022 (CN) 202210528479.0

(51) Int. Cl. G01N 33/68; B82Y 15/00; C12Q 1/6841; G01N 21/65; G01N 33/543

(52) U.S. Cl. CPC G01N 33/6803; C12Q 1/6841; G01N 21/658; G01N 33/54346; B82Y 15/00

(57) ABSTRACT

An in-situ membrane protein dimerization state detection kit and the use thereof. The kit comprises a membrane protein anchor chain, a recognition probe and a SERS tag. Catalytic hairpin assembly is triggered by means of adjacent hybridization of the membrane protein anchor chain, which carries an adaptor sequence, during membrane protein dimerization to form a networked assembly of the recognition probe and the SERS tag, thereby performing SERS detection and imaging on a highly specific event in a cell communication process, namely the membrane protein dimerization. The kit is suitable for high-sensitivity monitoring of intercellular signaling based on membrane protein dimerization in a complex cell microenvironment.

### TECHNICAL FIELD

The present invention belongs to the field of functional nanoprobe technology and biological detection, and specifically relates to an in situ detection kit for a membrane protein dimerization state and use thereof.

### RELATED ART

Multicellular organisms regulate their life activities through intercellular communication, that is, the information of a cell is transmitted to another cell through specific cell secretions to trigger a series of biochemical reactions, thus affecting the process of cellular biological functions. Membrane proteins play an important role in cell communication. Signaling is achieved through conformational changes triggered by the binding of membrane protein receptors to cytokines, which then activate intracellular signal transduction. In most cases, membrane protein receptors do not work alone, but instead signal by forming dimers or complexes. For example, upstream cytokine-induced receptor dimerization is considered as an important pathway to initiate intracellular signaling, and plays a key role in normal biological processes and cancer development. Reliable detection and monitoring of the dimerization process of membrane protein receptors is crucial for studying cell-to-cell communication and further biological behavior. There are many common proteins that function in a dimerized form. These proteins affect the growth and development, differentiation, formation of various tissues and organs, maintenance of tissues, and coordination of various physiological activities of organisms by regulating cell adhesion, movement, growth, and migration. For example, binding of the Met protein to its ligand hepatocyte growth factor (HGF) can induce Met dimerization, phosphorylate its substrate, activate downstream signaling pathways, and promote cell proliferation and migration, thus playing an important role in embryonic development and tumor occurrence and development. When TGF-β1 induces TβRII to form a dimer, TβRII itself will be phosphorylated, which then phosphorylates TβRI to make TβRI have serine/threonine kinase activity, and phosphorylates downstream receptor-related Smad signaling molecules, thus affecting the occurrence of epithelial-mesenchymal transition. In addition to the homodimer above, heterodimers such as HER2-HER3 can function to promote the proliferation of tumor cells.

The methods used in the prior art for detecting a membrane protein dimerization state are mainly based on traditional protein analysis methods. For example, the public document with DOI 10.1038/s41467-019-12241-2 uses immunohistochemical analysis for detection. However, these traditional detection methods can only detect the total amount of a membrane protein rather than the dimer state of the membrane protein, and cannot achieve in situ observation of the occurrence of membrane protein dimerization and the subsequent activation of cellular pathways.

### SUMMARY OF INVENTION

Surface enhanced Raman scattering (SERS) has good photostability, and is known as a powerful tool for reliable, easy-to-operate, and ultra-sensitive trace substance analysis. Its Raman characteristic peak is very narrow and it has the ability to detect multiple analytes simultaneously.

Therefore, for the above problems existing in the prior art, based on surface enhanced Raman scattering, the present invention provides an in situ detection kit for a membrane protein dimerization state and use thereof, so as to achieve SERS detection and in situ imaging for a highly specific event, i.e., membrane protein dimerization, in the process of cell communication. Catalytic hairpin self-assembly (CHA) is triggered through proximity hybridization of DNA single chains with aptamer sequences during membrane protein dimerization, to achieve networked assembly of a recognition probe and a SERS tag, so that simultaneous imaging of membrane protein dimerization, in the process of cell communication is realized. The kit of the present invention can monitor various intercellular signaling processes based on membrane protein dimerization in a complex cell microenvironment with high sensitivity and effectively distinguish different signaling processes by using different SERS tags.

### Technical Solution

In a first aspect, the present invention provides an in situ detection kit for a membrane protein dimerization state. The detection kit includes: a first membrane protein group anchoring chain, wherein the first membrane protein group anchoring chain comprises two first membrane protein anchoring chains respectively denoted as P1-T1 and P1-T2, which are respectively used to anchor two receptor protein monomers P1-1 and P1-2 on a first membrane protein, the P1-T1 and P1-T2 are capable of proximity hybridization during dimerization of the first membrane protein, and as shown in FIG. 1, the P1-T1 and P1-T2 each include four parts sequentially: an aptamer sequence, a free sequence, a hybridization sequence, and a trigger sequence; specifically, the aptamer sequences (denoted as P1-a) and the free sequences (denoted as P1-b) in P1-T1 and P1-T2 are the same, the hybridization sequence in P1-T1 is denoted as P1-c, and the trigger sequence therein is denoted as P1-d; the hybridization sequence in P1-T2 is denoted as P1-c′, and the trigger sequence therein is denoted as P1-d′; the hybridization sequences P1-c and P1-c′ are completely complementary to each other, and the hybridization sequence P1-c or P1-c′ has only 6-8 bases and preferably 7 bases, and such a sequence with only short complementary bases is difficult to form a stable double-stranded T-shaped structure in solution at room temperature; and the aptamer sequence P1-a can bind to the receptor protein monomer on the cell membrane; when the first membrane protein forms a dimer under the action of a dimerization inducer, the two membrane protein anchoring chains P1-T1 and P1-T2 can form a double-stranded T-shaped structure, i.e., a proximity hybridization structure, through complementary hybridization of the hybridization sequence P1-c or P1-c′; a recognition probe (RP), wherein the recognition probe is prepared by modifying AuNPs with a diameter ranging from 40 to 70 nm with a first RP hairpin, and the first RP hairpin is assembled from DNA sequences and is capable of specifically recognizing the trigger sequences to form a first complex with the double-stranded T-shaped structure; and a first SERS probe (SERS tag), wherein the first SERS probe is prepared by anchoring a first SERS hairpin and a first Raman molecule on AuNPs with a diameter ranging from 10 to 30 nm, and the first SERS hairpin is assembled from DNA sequences and has a sequence complementary to that of the first RP hairpin; the first Raman molecule is any one of 4-MBA, DTNB or 4-ATP.

Specifically, when the two membrane protein anchoring chains suffer from proximity hybridization during dimerization of the membrane protein, the non-hybridized portions of the trigger sequences thereof can serve as footholds to trigger the CHA reaction together; then, in the presence of the H2 sequence on the SERS tag, the complex can be opened to form a more stable H1-H2 double chain, thereby allowing the recognition probe and the SERS probe to assemble, and the released double-stranded T-shaped structure can bind to the recognition probe again on living cell membranes to trigger the next round of recognition probe and SERS probe assembly; as a result, through triggering of protein dimerization, a network nanostructure composed of AuNPs with two diameters of 10-30 nm and 40-70 nm is autonomously assembled around the cell membrane; and the enhanced Raman scattering of the Raman molecules can be detected through the strongly coupled surface plasmon resonance of the nanogaps between the particles of the network nanostructure.

Furthermore, if the detection method is used to detect the dimerization state of two membrane proteins simultaneously, the detection kit further includes a second membrane protein group anchoring chain for anchoring a second membrane protein, wherein the second membrane protein group anchoring chain also comprises two anchoring chains respectively denoted as P2-T1 and P2-T2, which are respectively used to anchor two receptor protein monomers P2-1 and P2-2 on a second membrane protein, the P2-T1 and P2-T2 are also capable of proximity hybridization during dimerization of the second membrane protein to form a double-stranded T-shaped structure, and the P2-T1 and P2-T2 also each include four parts: an aptamer sequence, a free sequence, a hybridization sequence, and a trigger sequence; also, the aptamer sequences (denoted as P2-a) and the free sequences (denoted as P2-b) in P2-T1 and P2-T2 are the same, the hybridization sequence in P2-T1 is denoted as P2-c, and the trigger sequence therein is denoted as P2-d; the hybridization sequence in P2-T2 is denoted as P2-c′, the trigger sequence therein is denoted as P2-d′, and the hybridization sequences P2-c and P2-c′ are complementary to each other, and the hybridization sequence P2-c or P2-c′ also has only 6-8 bases and preferably 7;
the recognition probe (RP) is a dual recognition probe (dual-RP), wherein the dual recognition probe is prepared by modifying the AuNPs with a diameter ranging from 40 to 70 nm with two different DNA hairpins simultaneously, that is to say, the recognition probe further includes a second RP hairpin; the second RP hairpin is assembled from DNA sequences, and is capable of specifically recognizing the trigger sequences P2-dd′ to form a second complex.

It should be noted that, obviously, in order to ensure the specificity in simultaneous detection of the dimerization state of two membrane proteins, the DNA sequences of the first RP hairpin and the second RP hairpin must avoid hybridization with each other, do not form a stable hybridization relationship with each other, and are capable of binding to P1-dd′ and P2-dd′ respectively to open their hairpin structures.

Preferably, the sequences of the first RP hairpin and the second RP hairpin are as follows, respectively:

first RP hairpin:
5′-SH-TTTTTACACGAACACAGGGATACAGCCGT
GACAAGTAGCTCTGTATCCCTGTGT-3′;
second RP hairpin:
5′-SH-TTTTTCGAGGACGATGTAGACGAAGCCCG
ACGTTACTTATCTTCGTCTACATCG-3′.

The detection kit further comprises a second SERS probe, wherein the second SERS probe is prepared by anchoring a second SERS hairpin and a second Raman molecule on the 15 nm AuNPs, the second SERS hairpin is assembled from DNA sequences and has a sequence complementary to that of the second RP hairpin; the second Raman molecule is selected from any one of 4-MBA, DTNB or 4-ATP, but is different from the first Raman molecule.

In a second aspect, the present invention provides use of the above detection kit in simultaneous detection of an Met protein dimerization state and a TβRII protein dimerization state, wherein an enhanced 4-MBA signal during dimerization of the Met protein and an enhanced DTNB signal when dimerization of the TβRII protein can be obtained, and this use method has been proved to specifically detect and image Met and TβRII protein dimerization, thereby achieving the monitoring of two cell communications; when the above detection kit is used for the simultaneous detection of the Met protein dimerization state and the TβRII protein dimerization state, the recognition probe (RP) is a dual recognition probe (dual-RP), which can be used for simultaneous recognition of the two membrane proteins Met and TβRII protein; the first SERS probe is an Met-SERS tag; the second SERS probe is a TβRII-SERS tag; preferably, the molar ratio of the dual-RP, the Met-SERS tag and the Tgt-SERSRS tag is 1:25:25; and the use includes the following steps: first, incubating cells to be detected with anchoring chains T-Met-1, T-Met-2, T-TβRII-1 and T-TβRII-2 in a medium, so that the anchoring chains bind to the corresponding Met or TβRII proteins on the cell membrane; washing three times with PBS, and then incubating the cells with the dual-RP, the Met-SERS tag and the TβRII-SERS tag; washing the cells with PBS to remove free and non-specifically deposited nano-probes, and then performing SERS imaging of the protein dimers on living cells.

As a preferred embodiment of the use of the present invention, the preparation of the dual recognition probe dual-RP includes the following steps: incubating the first RP hairpin, i.e. H1-Met, and the second RP hairpin, i.e. H1-TβRII, together with 50 nm AuNPs at room temperature for 6-12 h, and then aging, i.e. adding a NaCl solution four times with an interval of 30 min each time to make the final NaCl concentration of 0.2 M, shaking the mixture gently at room temperature for 6-12 h, and finally centrifuging and washing three times with PBS (4000 rpm, 20 min); resuspending the precipitate in PBS to obtain the dual-RP; further preferably, the molar ratio of H1-Met, H1-TβRII and 50 nm AuNPs is 5000:5000:1-7000:7000:1.

As a preferred embodiment of the use of the present invention, the preparation of the first SERS probe Met-SERS tag includes the following steps: incubating the first SERS hairpin H2-Met with 15 nm AuNPs at room temperature for 6-12 h, aging as described above, and then incubating the first Raman molecule 4-MBA with H2-Met-modified AuNPs at room temperature for 3 h, centrifuging and washing three times with PBS (12000 rpm, 20 min), and then resuspending the precipitate in PBS to obtain the Met-SERS tag; preferably, the molar ratio of H2-Met to 15 nm AuNPs is 400:1-600:1; the molar ratio of 4-MBA to H2-Met modified 15 nm AuNPs is 1500:1-2000:1.

As a preferred embodiment of the use of the present invention, the preparation of the second SERS probe TβRII-SERS tag includes the following steps: incubating the second SERS hairpin H2-TβRII with 15 nm AuNPs at room temperature for 6-12 h, aging, and then incubating the second Raman molecule DTNB with H2-TβRII-modified AuNPs at room temperature for 3 h; centrifuging and washing three times with PBS (12000 rpm, 20 min), and then resuspending the precipitate in PBS to obtain the TβRII-SERS tag; preferably, the molar ratio of H2-TβRII to 15 nm AuNPs is 400:1-600:1; the molar ratio of DTNB to H2-TβRII-modified 15 nm AuNPs is 750:1-1000:1.

As shown in FIG. 2A, the Met-SERS tag is prepared by labeling 15 nm AuNPs with H2-Met and the Raman molecule 4-MBA together, and the dual-RP is prepared by modifying 50 nm AuNPs with H1-Met and H1-TβRII simultaneously. As shown in FIG. 2B, the specially designed T-Met-1 and T-Met-2 only include seven complementary bases, and are difficult to form a stable T-Met-1-2 double-stranded T-shaped structure in solution at room temperature. The aptamer sequences in T-Met-1 and T-Met-2 when incubated with target cells can bind to the receptor protein Met monomer on the cell membrane. Once the Met protein forms a dimer under the action of the dimerization inducer HGF, T-Met-1 and T-Met-2 can hybridize through the seven complementary bases to form a T-Met-1-2 double-stranded T-shaped structure, that is, proximity hybridization occurs. The unhybridized portions of T-Met-1 and T-Met-2 can serve as footholds to trigger the CHA reaction together. As shown in FIG. 2B, the T-shaped structure can hybridize with the H1-Met used to label the dual-RP to form an H1-T-Met-1-2 complex. The CHA reaction in the present invention is a CHA-based dynamic assembly of AuNPs triggered by proximity hybridization on living cell membranes; then, with the help of H2 on the Met-SERS tag, the H1-T-Met-1-2 complex can be opened to form a more stable H1-H2-Met double chain, allowing assembly of the dual-RP and Met SERS tag, and the released T-Met-1-2 double-stranded T-shaped structure can bind to the dual-RP again on living cell membranes to trigger the next round of assembly. Therefore, through triggering of protein dimerization, a network aggregate including 15 nm and 50 nm AuNPs (i.e., 15Au-50Au network nanostructure) is autonomously assembled around the cell membrane. Meanwhile, the enhanced Raman scattering of the Raman molecules can be detected due to the strongly coupled surface plasmon resonance of the nanogaps between the particles of the 15Au-50Au network nanostructure. Therefore, the detection kit of the present invention, when used to detect the dimerization state of the membrane proteins, combines the advantages of high-specificity recognition, cyclic amplification of CHA and dynamic assembly of AuNPs, so that an effective way can be provided to conveniently observe protein dimerization on living cell membranes. In addition, as shown in FIG. 2A, with the dual-RP constructed by labeling 50 nm AuNPs with H1-Met and H1-TβRII together and the TβRII SERS tag prepared by modifying 15 nm AuNPs with DTNB Raman molecules and H2-TβRII, the dimerization of two types of cell membrane proteins can be observed simultaneously, as shown in FIG. 2C. By triggering catalytic hairpin self-assembly (CHA) through proximity hybridization of DNA single chains with aptamer sequences during dimerization of membrane proteins, the detection kit achieves networked assembly of a dual recognition probe (dual-RP) and an SERS tag, thereby realizing simultaneous imaging of dimerization of two cell membrane proteins.

Beneficial effects: The detection kit of the present invention can be used for SERS detection and imaging for a highly specific event, i.e., membrane protein dimerization, in the process of cell communication. Catalytic hairpin self-assembly (CHA) is triggered through proximity hybridization of DNA single chains with aptamer sequences during dimerization of membrane proteins, so as to achieve networked assembly of a recognition probe (dual-RP) and an SERS tag, thereby realizing imaging of dimerization of at least one cell membrane protein. The detection kit can monitor intercellular signaling based on membrane protein dimerization in a complex cellular microenvironment with high sensitivity, and can also monitor at least two types of intercellular signaling based on membrane protein dimerization by using different Raman molecules.

The detection kit provided by the present invention can achieve the monitoring of intercellular signaling based on membrane protein dimerization in a complex cellular microenvironment, and greatly improves the sensitivity compared with existing methods, thereby enabling cell communication monitoring in a cell culture environment that is previously difficult to accomplish. Compared with traditional immunoassays, in which simple protein blotting methods are difficult to clearly distinguish various cell communications due to the complexity of the protein phosphorylation process involved in cell communication and the intersection of various signal pathways, the present invention realizes the detection of two types of intercellular signaling based on membrane protein dimerization, has high specificity, and can distinguish various cell communications in complex systems. Meanwhile, the SERS sensing technology used in the present invention can overcome the shortcomings of easy photobleaching, complex background fluorescence and difficulty in achieving multi-channel imaging in fluorescence technology.

### BRIEF DESCRIPTION OF DRAWINGS

FIG. 1 is a schematic diagram of the structures of the two membrane protein anchoring chains P1-T1 and P1-T2 in the detection kit of the present invention;

FIG. 2A is a schematic diagram of the assembly of the dual recognition probe, the first SERS probe, and the first SERS probe when the detection kit of the present invention is used for simultaneous detection of the Met protein dimerization state and TβRII protein dimerization state;

FIG. 2B is a schematic diagram of the detection process of the Met protein dimerization state in the early stage when the detection kit of the present invention is used for simultaneous detection of the Met protein dimerization state and TβRII protein dimerization state;

FIG. 2C is a schematic diagram of the detection process of the TβRII protein dimerization state in the later stage when the detection kit of the present invention is used for simultaneous detection of the Met protein dimerization state and TβRII protein dimerization state;

FIG. 3 is a TEM characterization of 15 nm AuNPs and 50 nm AuNPs used in Example 1 of the present invention;

FIG. 4 is a gel electrophoresis characterization diagram of simulation of the working mechanism of protein dimerization in Example 2 of the present invention;

FIG. 5A is absorption spectra of the 15 nm AuNPs, the Met-SERS tags, the TβRII-SERS tags, the 50 nm AuNPs and the dual-RPs in Example 3 of the present invention and absorption peak wavelengths thereof;

FIG. 5B is absorption spectra of the dual-RPs, Met-SERS tags, TβRII-SERS tags and mixtures thereof under stimulation of different targets in Example 3 of the present invention and absorption peak wavelengths thereof;

FIG. 5C is a scanning electron micrograph of the dual-RPs, Met-SERS tags, TβRII-SERS tags and mixtures thereof under stimulation of different targets in Example 3 of the present invention;

FIG. 5D is a transmission electron micrograph of the dual-RPs, Met-SERS tags, TβRII-SERS tags and mixtures thereof under stimulation of different targets in Example 3 of the present invention;

FIG. 5E is a comparison diagram of the particle sizes of the dual-RPs, Met-SERS tags, TβRII-SERS tags and mixtures thereof under stimulation of different targets in Example 3 of the present invention;

FIG. 5F is the particle sizes of the dual-RPs, Met-SERS tags, TβRII-SERS tags and mixtures thereof under stimulation of different targets in Example 3 of the present invention;

FIG. 6 is distribution of the electromagnetic field before and after probe assembly simulated by FDTD in Example 3 of the present invention;

FIG. 7 is the MTT cytotoxicity test of the dual-RPs, Met-SERS tags and TβRII-SERS tags in Example 4 of the present invention;

FIG. 8 is the visualization imaging of Met protein dimerization over time in Example 4 of the present invention, wherein Panel A is SERS imaging, Panel B is dark field imaging, Panel C is the statistical result of the SERS signal intensity in Panel A, and Panel D is the statistical result of the number of bright spots of large particles assembled in Panel B;

FIG. 9 is the SERS imaging of Met protein dimerization on DU145 cells in Example 4 of the present invention;

FIG. 10 is the results of the respective SERS imaging for Met and TβRII protein dimerization and the protein blotting analysis of downstream protein phosphorylation on DU145 cells in Example 4 of the present invention;

FIG. 11 is the results of the simultaneous SERS imaging for Met and TβRII protein dimerization and the protein blotting analysis of downstream protein phosphorylation on DU145 cells in Example 5 of the present invention;

FIG. 12 is the SEM image of the detection of DU145 cells in Example 5 of the present invention;

FIG. 13 is the SERS imaging of cell communication between DU145 cells and WPMY-1 cells in Example 6 of the present invention, wherein Panel A is a schematic diagram of the experiment, Panel B is the results of the protein blotting analysis of protein phosphorylation, and Panel C is the SERS imaging result of protein dimerization; and

FIG. 14 is the imaging of communication between DU145 cells and BMSCs cells in Example 6 of the present invention.

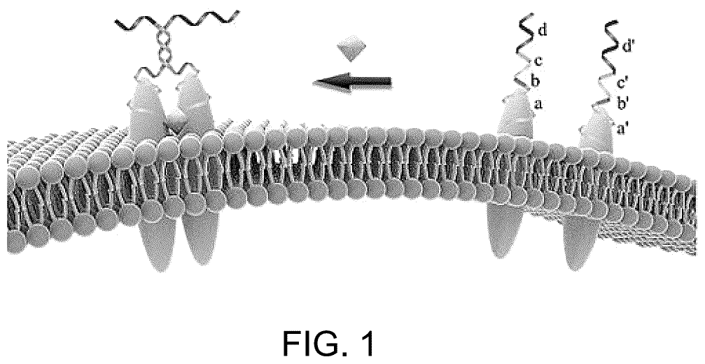

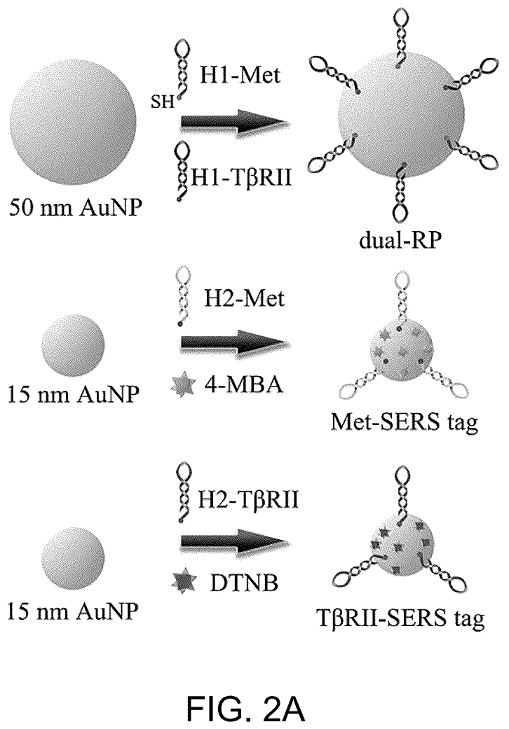

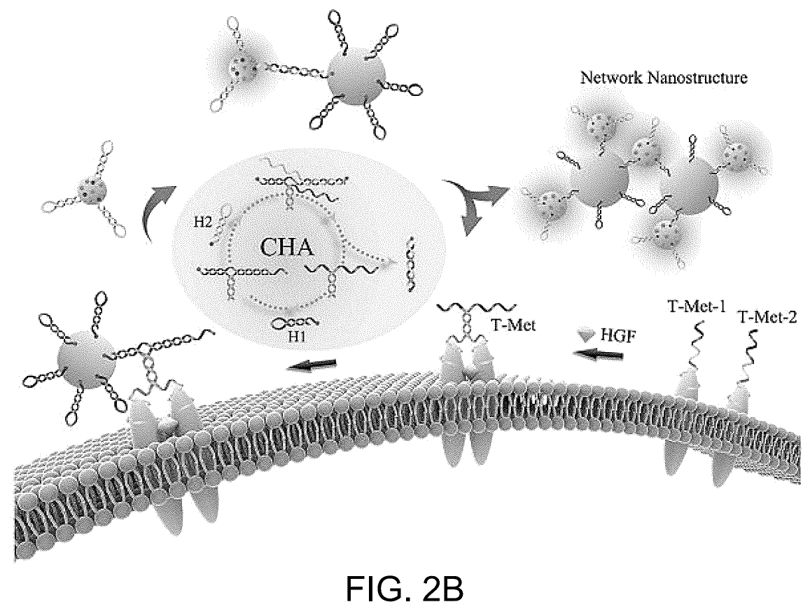

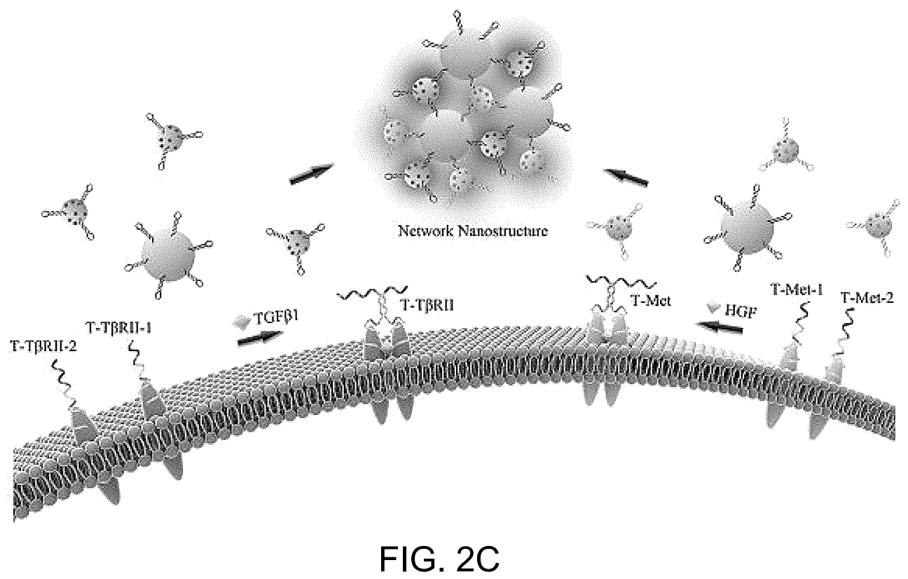

### DESCRIPTION OF EMBODIMENTS

The present invention is further described in detail in conjunction with the following specific examples and accompanying drawings. The following examples are only illustrative, and the protection content of the present invention is not limited thereto. The nucleotide chains of the present invention are all synthesized and provided by Sangon Biotech (Shanghai) Co., Ltd.

### Design of Nucleotide Chain Sequence

Taking the kit for simultaneous detection of Met protein dimerization and TβRII protein dimerization as an example, two sets of preferred sequence designs of DNA sequences involved in the first membrane protein group anchoring chain, the second membrane protein group anchoring chain, the dual recognition probe, the first SERS probe and the second SERS probe in the kit are provided.

The first membrane protein group anchoring chain T-Met-1 and T-Met-2 can trigger proximity hybridization when an Met protein dimerizes, while the second membrane protein group anchoring chain T-TβRII-1 and T-TβRII-2 can trigger proximity hybridization when a TβRII protein dimerizes, wherein the dual recognition probe (dual-RP) is prepared by modifying 50 nm AuNPs with DNA hairpins, i.e., the first RP hairpin H1-Met and the second RP hairpin H1-TβRII, the first SERS probe Met-SERS tag is prepared by anchoring DNA hairpins, i.e., the first SERS hairpin H2-Met and the Raman molecule 4-MBA on 15 nm AuNPs, and the second SERS probe TβRII-SERS tag is prepared by anchoring DNA hairpins, i.e., the second SERS hairpin H2-TβRII and the Raman molecule DTNB on 15 nm AuNPs.

Specific sequence designs include but are not limited to the following two sets of preferred sequences:

| Se- | Design of the | Design of the |
|---|---|---|
| quence | first set of | second set of |
| name | sequences. | sequences |
| T-Met- | SEQ ID NO: 1 | SEQ ID NO: 9 |
| 1 | 5′-TGGATGGTAGCTCGGTC | 5′-TGGATGGTAGCTCGGTC |
| | GGGGTGGGTGGGTTGGCAAG | GGGGTGGGTGGGTTGGCAAG |
| | TCTTTTTTTTTTTTTTTTAG | TCTTTTTTTTTTTTTTTTTC |
| | TGAGGTTCGTGT-3′ | AGAGCCTTGCTCT-3′ |
| T-Met- | SEQ ID NO: 2 | SEQ ID NO: 10 |
| 2 | 5′-CTGTATCCCTGTGTCCC | 5′-GTCTATGGGTCTCTGGG |
| | TCACTTTTTTTTTTTTTTTT | TCTGATTTTTTTTTTTTTTT |
| | TGGATGGTAGCTCGGTCGGG | TGGATGGTAGCTCGGTCGGG |
| | GTGGGTGGGTTGGCAAGTC | GTGGGTGGGTTGGCAAGTC |
| | T-3′ | T-3′ |
| T- | SEQ ID NO: 3 | SEQ ID NO: 11 |
| TβRII- | 5′-ACATTGCTGCGTGATCG | 5′-ACATTGCTGCGTGATCG |
| 1 | CCTCACATGGGTTTGTCTGG | CCTCACATGGGTTTGTCTGG |
| | TCGATTTGGAGGTGGTGGGT | TCGATTTGGAGGTGGTGGGT |
| | GGCTTTTTTTTTTTTTTTTT | GGCTTTTTTTTTTTTTTTTT |
| | CTCATGCTTCCTCG -3′ | GAGATCGTTGGTGC-3′ |
| T- | SEQ ID NO: 4 | SEQ ID NO: 12 |
| TβRII- | 5′-CTTCGTCTACATCGTGC | 5′-GTTGCTGTAGATGCTCG |
| 2 | ATGAGTTTTTTTTTTTTTTT | ATCTCTTTTTTTTTTTTTTT |
| | ACATTGCTGCGTGATCGCCT | TACATTGCTGCGTGATCGCC |
| | CACATGGGTTTGTCTGGTCG | TCACATGGGTTTGTCTGGTC |
| | ATTTGGAGGTGGTGGGTGGC | GATTTGGAGGTGGTGGGTGG |
| | -3′ | C-3′ |
| H1- | SEQ ID NO: 5 | SEQ ID NO: 13 |
| Met | 5′-SH-TTTTTACACGAACA | 5′-SH-TTTTTAGAGCAAGA |
| | CAGGGATACAGCCGTGACAA | GACCCATAGACGGCTCAGAA |
| | GTAGCTCTGTATCCCTGTGT | CTACGTGTCTATGGGTCTCT |
| | -3′ | -3′ |
| H2- | SEQ ID NO: 6 | SEQ ID NO: 14 |
| Met | 5′-SH-TTTTTGGATACAGA | 5′-SH-TTTTTCCATAGACA |
| | GCTACTTGTCACGGCTGTAT | CGTAGTTCTGAGCCGTCTAT |
| | CCCTGTGTGACAAGTAGCTC | GGGTCTCTCAGAACTACGTG |
| | -3′ | -3′ |
| H1- | SEQ ID NO: 7 | SEQ ID NO: 15 |
| TβRII | 5′-SH-TTTTTCGAGGACGA | 5′-SH-TTTTTGCACCAGCA |
| | TGTAGACGAAGCCCGACGTT | TCTACAHCAACHHHCAHCTT |
| | ACTTATCTTCGTCTACATCG | AGTTATGTTGCTGTAGATGC |
| | -3′ | -3′ |
| H2- | SEQ ID NO: 8 | SEQ ID NO: 16 |
| TβRII | 5′-SH-TTTTTAGACGAAGA | 5′-SH-TTTTTACAGCAACA |
| | TAAGTAACGTCGGGCTTCGT | TAACTAAGCTGCCCGTTGCT |
| | CTACATCGACGTTACTTATC | GTAGATGCAGCTTAGTTATG |
| | -3′ | -3′ |

### Example 1 Preparation of Dual Recognition Probe Dual-RP, First SERS Probe and Second SERS Probe

The nucleotide chains H1-Met sequence involved in Example 1 is shown in SEQ ID NO: 5, the H2-Met sequence is shown in SEQ ID NO: 6, the H1-TβRII sequence is shown in SEQ ID NO: 7, and the H2-TβRII sequence is shown in SEQ ID NO: 8.

1. Preparation of dual recognition probe dual-RP: 1) A 50 μL solution containing 10 μM H1-Met and H1-TβRII was incubated with 500 μL of 50 nm AuNPs (0.075 nM) at room temperature for 12 h, and then aged, i.e., a NaCl solution was added four times with an interval of 30 min each 10 time to make the final NaCl concentration of 0.2 M, and the mixture was gently shaken at room temperature for 12 h; 2) the mixture was centrifuged and washed three times with PBS (4000 rpm, 20 min). The precipitate was resuspended in 50 μL PBS to obtain the dual-RP and stored at 4° C. for further use.

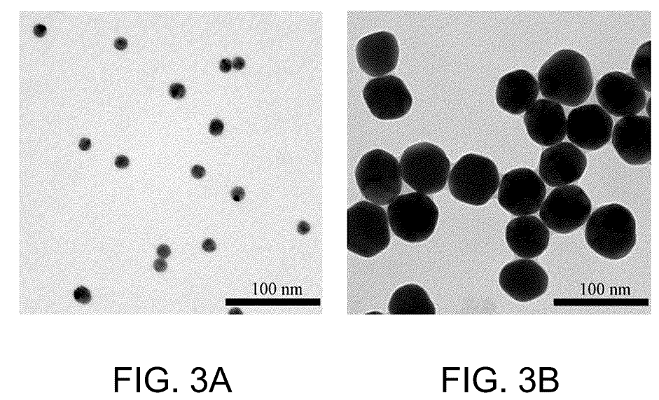

2. Preparation of the first SERS probe Met-SERS tag: 1) H2-Met (50 μM, 10 μL) was incubated with 500 μL of 15 nm AuNPs (2.325 nM, Panel A of FIG. 3) at room temperature for 12 h, and aged as described above; 2) 20 μL of 100 μM 4-MBA was incubated with 500 μL of H2-Met-modified AuNPs at room temperature for 3 h; 3) the mixture was centrifuged and washed three times with PBS (12000 rpm, 20 min), and then the precipitate was resuspended in 50 μL PBS to obtain the Met-SERS tag and stored at 4° C. for further use.

3. Preparation of the second SERS probe TβRII-SERS tag: 1) H2-TβRII (50 μM, 10 μL) was incubated with 500 μL of 15 nm AuNPs (2.325 nM, Panel B of FIG. 3) at room temperature for 12 h, and aged as described above;
2) 10 μL of 100 μM DTNB was incubated with 500 L of H2-Met-modified AuNPs at room temperature for 3 h; 3) the mixture was centrifuged and washed three times with PBS (12000 rpm, 20 min), and then the precipitate was resuspended in 50 μL PBS to obtain the TβRII-SERS tag and stored at 4° C. for further use.

### Example 2 Characterization of the Mechanism of Proximity Hybridization Reaction During Protein Dimerization

In order to verify the correctness of the mechanism, a 10% PAGE gel was used for a gel electrophoresis experiment. A 5 μL of DNA sample was mixed well with a 1 μL of 6×DNA loading buffer, run at 80 V for 80 min, and then imaged.

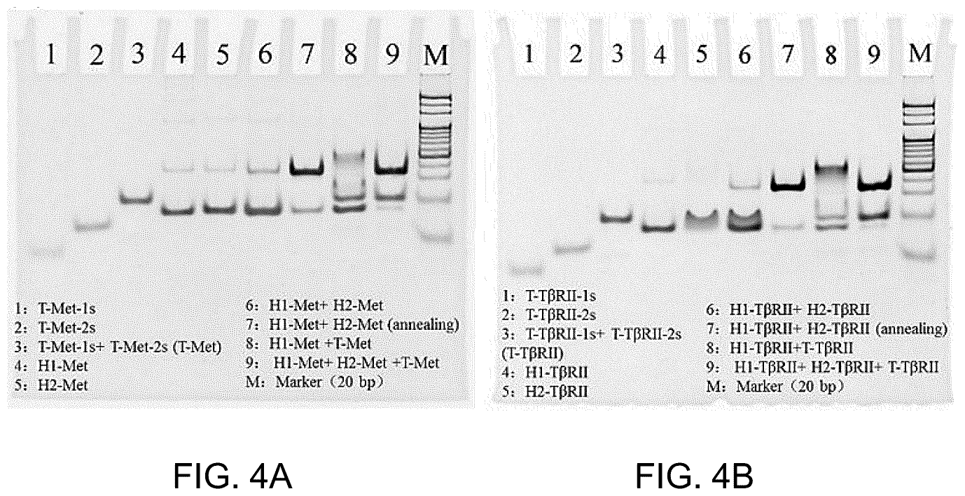

FIG. 4 shows a gel electrophoresis characterization diagram of simulation of the working mechanism of protein dimerization in the present invention. In order to simulate the T-Met-1-2 double-stranded T-shaped structure (or the T-TβRII-1-2 double-stranded T-shaped structure) formed by proximity hybridization during protein dimerization, the complementary base numbers of the hybridization sequences of T-Met-1 and 2 (or T-TβRII-1 and 2) were increased to obtain T-Met-1s and 2s (or T-TβRII-1s and 2s), with specific simulation sequences as follows:

T-Met-1s like SEQ ID NO: 17 is
5′-AAAAAAAAAAAAAGTGAGGTTCGTGT-3′
T-Met-2s like SEQ ID NO: 18 is
5′-CTGTATCCCTGTGTCCCTCACTTTTTTTTTTTTT-3′
T-TβRII-1s like SEQ ID NO: 19 is
5′-AAAAAAAAAAAACTCATGCTTCCTCG-3′
T-TβRII-2s like SEQ ID NO: 20 is
5′-CTTCGTCTACATCGTGCATGAGTTTTTTTTTTTT-3′

In Panel A of FIG. 4, T-Met-1s (Lane 1) and T-Met-2s (Lane 2) can form a DNA double chain (Lane 3, T-Met) with high yield. The electrophoresis results of H1-Met and H2-Met in Lanes 4 and 5 show that H1-Met and H2-Met with hairpin structures can protect themselves from hybridization at room temperature (Lane 6), and can form an H1-H2-Met double chain after being annealed (Lane 7). The band in Lane 8 indicates that H1-Met can hybridize with T-Met and further induce the formation of the H1-H2-Met double chain with the release of T-Met in Lane 9. Panel B of FIG. 4 characterizes the nucleic acid hybridization reaction in response to the dimerization of the TβRII protein, and the feasibility of the reaction is also confirmed by the formation of the H1-H2 TβRII double chain triggered by the hybridization double chain (T-TβRII) of T-TβRII-1s and T-TβRII-2s.

### Example 3 Assembly of AuNP Probes in Buffer to Form Network Nanostructures

Equal volume of 30 μL of the Met-SERS tags and the TβRII-SERS tags in Example 1 were mixed, into which then 60 μL of the dual RPs containing 50 nM T simulation chains, i.e., T-Met-1s, T-Met-2s, T-TβRII-1s and T-TβRII-2s, were added, and then incubated with gentle shaking at 37° C. for 2 h to form 15Au-50Au network nanostructures assembled by 15 nm AuNPs and 50 nm AuNPs. The 15Au-50Au network nanostructures without centrifuging and washing were directly characterized by absorption spectroscopy, TEM and SEM imaging. The SERS spectrum of the 15Au-50Au network nanostructure was collected by a Renishaw system. The nanoparticles were measured by DLS after being centrifuged and washed (3000 rpm, 20 min).

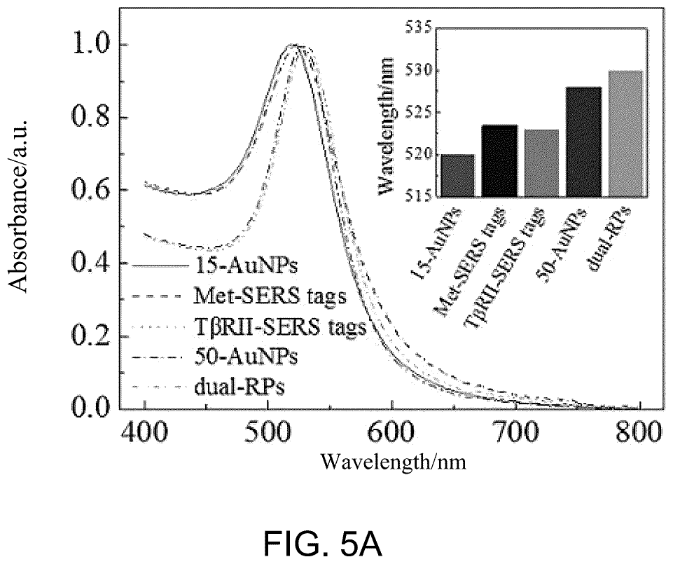

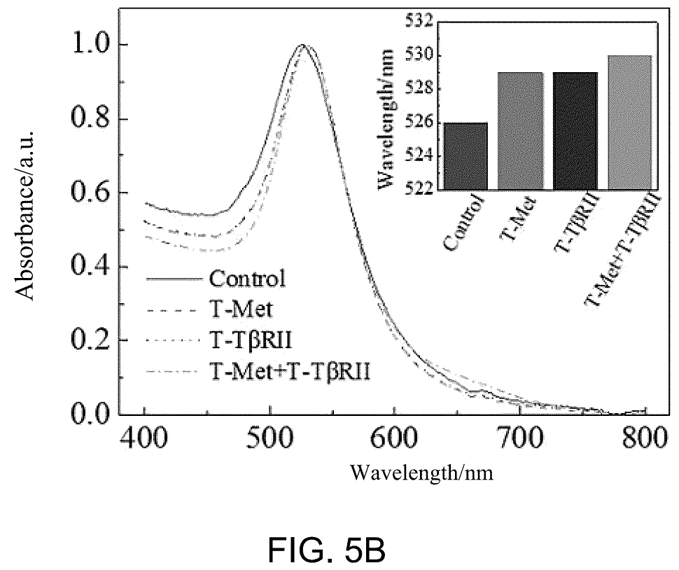

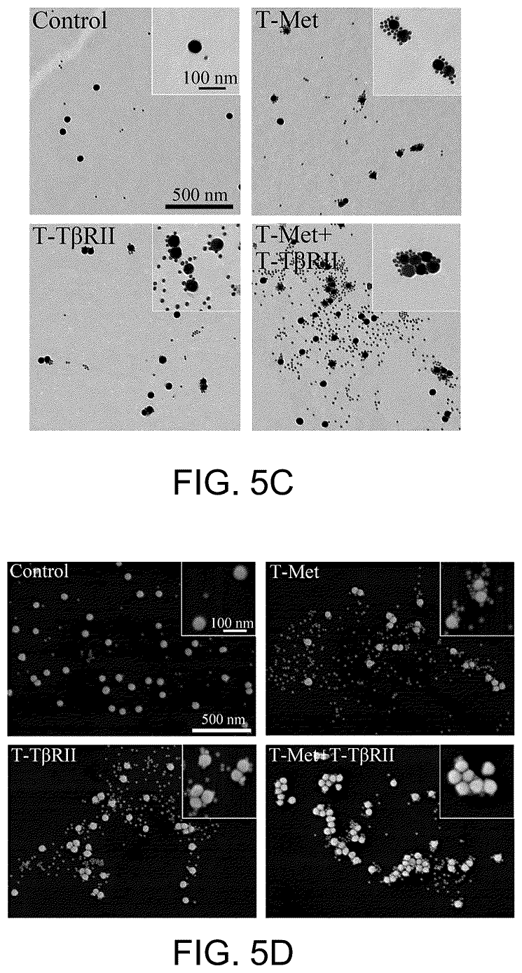

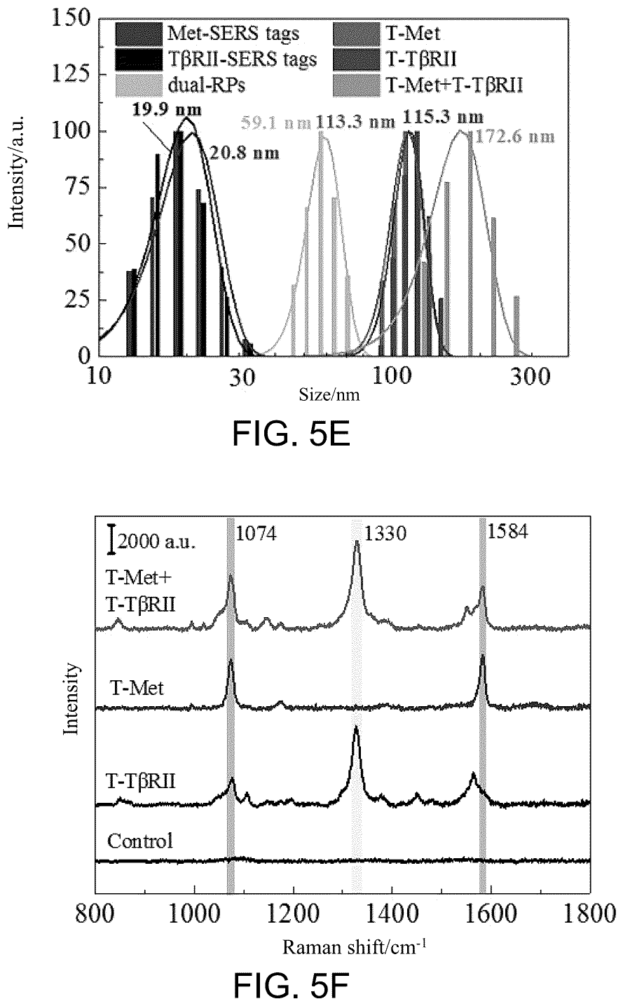

The assembly of AuNP network nanostructures due to protein dimerization and subsequent SERS sensing were verified by characterizing the morphology and optical properties of AuNPs. After surface modification with H1-Met and H1-TβRII, as shown in the curve of dual-RPs in FIG. 5A, the surface plasmon resonance (SPR) peak of 50 nm AuNPs at 528 nm was red-shifted to 530 nm. Likewise, after surface modification with 4-MBA and H2-Met (or DTNB and H2-TβRII), as shown in the curves of Met-SERS tags and TβRII-SERS tags in FIG. 5A, the SPR of 15 nm AuNPs was red-shifted from 520 nm to 523.5 nm (or 523 nm), indicating that the Met-SERS tag (or TβRII-SERS tag) was successfully prepared. The SPR of the mixture of the dual-RP, Met-SERS tag and TβRII-SERS tag was at 526 nm, as shown in the curve of Control in FIG. 5B, which is between that of the dual-RP and SERS tag. As shown in the TEM image of the Control group in FIG. 5C, the monodispersity of 15 nm and 50 nm AuNPs is good. As shown in FIG. 5E, the hydrated diameters of the dual-RP, Met-SERS tag and TβRII-SERS tag are 59.1, 20.8 and 19.9 nm, respectively. As shown in the Control group in FIG. 5F, the SERS signal of the mixture of the dual-RP, Met-SERS tag and TβRII-SERS tag is weak. In the presence of T-Met (i.e., T-Met 1s and 2s) or T-TβRII (i.e., T-TβRII 1s and 2s), the mixture of the dual-RP, Met-SERS tag, and TβRII-SERS tag has an SPR at 529 nm, as shown in the curves of T-Met or T-TβRII in FIG. 5B, and has a hydrodynamic diameter (T-Met or T-TβRII) of about 113.3 nm (or 115.3 nm), as shown in FIG. 5E. The size of the 15Au-50Au network nanostructures was relatively smaller as observed in the TEM image of the T-Met or T-TβRII group in FIG. 5C. When the mixture contains T-Met, T-TβRII, the dual-RP, the Met-SERS tag, and the TβRII-SERS tag, it has an SPR shifted to 530 nm, as shown in the corresponding curve of T-Met+T-TβRII in FIG. 5B, and has a hydrated particle size increased to 172.6 nm, as shown in the corresponding curve of T-Met+T-TβRII in FIG. 5E, which is due to the larger 15Au-50Au network nanostructures assembled in the solution. As shown in FIG. 5D, the SEM images obtained by dropping different solutions on the silicon wafer are consistent with the TEM characterization. These results confirm that proximity hybridization induced by membrane protein dimerization can effectively trigger AuNP assembly. FIG. 5F is the SERS characterization of the 15Au-50Au network nanostructures, wherein the network nanostructures show enhanced 4-MBA and DTNB signals for the case of simulating simultaneous dimerization of Met and TβRII proteins, in which 1074 cm-1 and 1584 cm-1 correspond to 4-MBA; 1330 cm-1 corresponds to DTNB, and for the case of simulating single dimerization of Met or TβRII protein, only the single SERS signal of its corresponding SERS tag shows an enhancement effect.

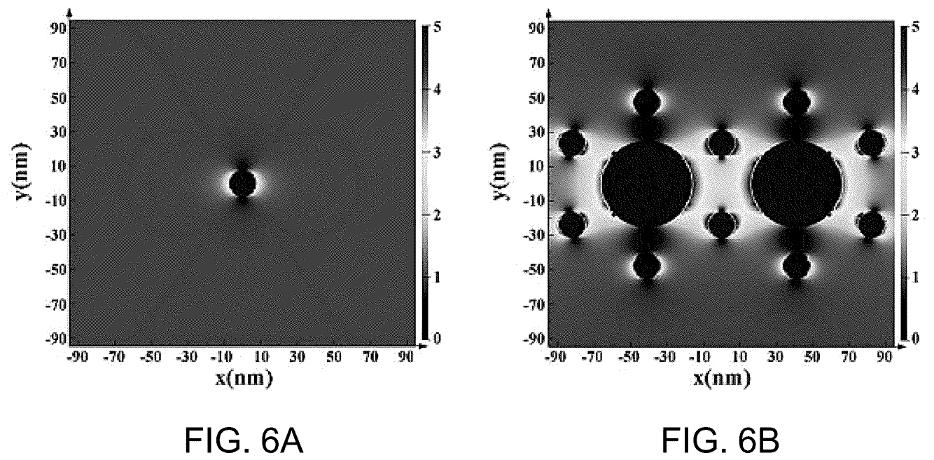

In addition, the local electromagnetic fields of the nanostructures were theoretically simulated by FDTD (Lumerical FDTD Solutions 2018). For the networked nanostructures, the gap distance between AuNPs was set to 10 nm according to the length of the DNA chain assembling the two particles. A total-scattered-field light source with an excitation wavelength of 633 nm was used to calculate the local electromagnetic properties of the AuNP networked nanostructures. The mesh size of the simulation area was set to 0.5 nm. FDTD simulation theoretically has verified that the electromagnetic field of the networked nanostructure is significantly enhanced. This is because the electromagnetic field intensity of the nanogap between adjacent AuNPs, as shown in Panel A of FIG. 6, is much stronger than that of a single AuNP, as shown in Panel B of FIG. 6. The above results demonstrate the feasibility of visual detection of membrane protein dimerization based on SERS technology.

### Example 4 Unit SERS Imaging of Membrane Protein Dimerization in DU145 Cells

1) Culture of test cells: Human prostate stromal myofibroblast cell line (WPMY-1) and human prostate cancer cell line (DU145) were purchased from Shanghai Institutes for Biological Sciences, Chinese Academy of Sciences (Shanghai, China). Bone marrow mesenchymal stem cells (BMSCs) were purchased from Cyagen (Suzhou, China). The cell medium was a DMEM medium supplemented with 10% FBS and 1% antibiotics (penicillin/streptomycin, 100 U/mL) (FBS-10% DMEM), and all the cells were cultured in a cell culture incubator according to the provided protocols.

2) Cytotoxicity test: The cytotoxicity of the nanoprobes was evaluated by an MTT experiment, and the specific experiment process was as follows: 5×10³ cells were inoculated in each well of a 96-well plate and cultured for 24 h; then, a series of concentrations of the dual-RPs, Met-SERS tags and TβRII-SERS tags were added to the wells and cultured for 24 h; cells that underwent the same treatment but without the addition of any probes were used as control.

3) Detection and SERS imaging of membrane protein dimerization: The T-Met-1 sequence as shown in SEQ ID NO: 1, the T-Met-2 sequence as shown in SEQ ID NO: 2, the T-TβRII-1 sequence as shown in SEQ ID NO: 3, and the T-TβRII-2 sequence as shown in SEQ ID NO: 4 were used; and the specific detection process of membrane protein dimerization provided in this example was as follows: in 1 mL of a DMEM medium without FBS, the cells were incubated with 100 nM T (including T-Met-1, T-Met-2, T-TβRII-1 and T-TβRII-2) for 10 min to allow T binding to the corresponding Met or TβRII protein on the cell membrane. After three PBS washes, the cells were incubated with 0.02 nM of the dual-RPs and 0.5 nM of the SERS tags (including the Met-SERS tags and TβRII-SERS tags). After 3 h, the cells were washed three times with PBS to remove free and nonspecifically deposited nanoprobes, and then SERS imaging of the protein dimers was performed on living cells.

In order to observe the membrane protein dimers on living cells, Met and TβRII-positive human prostate cancer cells DU145 were specifically selected as a cell model. DU145 cells were cultured in a DMEM medium and grown to a density of about 60%. Then the cells were starved in a DMEM medium without FBS and double antibodies for 24 h and subjected to three treatments. 1) No treatment: The cells were resuspended directly with trypsin. 2) Membrane protein dimerization induction treatment: The membrane protein dimerization inducer HGF (100 ng/ml) or TGFβ1 (20 ng/mL) was added to the DMEM culture medium, the mixture was incubated for 30 min, and the cells were resuspended with trypsin. 3) Inhibitor treatment: HGF (100 ng/ml) was co-incubated with an HGF inhibitor (2 μg/mL) for 1 h, TGFβ1 (20 ng/mL) was co-incubated with a TGFβ inhibitor (10 μg/mL) for 1 h, and then the mixture was incubated with the cells for 30 min, and the cells were resuspended with trypsin. The three cells treated by the different methods were subjected to subsequent SERS detection according to the detection steps above for membrane protein dimerization.

4) Time-dependent SERS and dark-field imaging of protein dimerization on living cells: To monitor protein dimerization on living cells, single dimerization of the Met protein was used as a model. DU145 cells (1×10⁵ cells/dish) were cultured with 100 ng/mL HGF in 1 mL DMEM without FBS for 30 min. Afterwards, the cells were incubated with 100 nM T-Met-1 and T-Met-2 for 10 min. The mixture was washed three times with PBS, into which then the dual-RPs and the Met-SERS tags were added, and the cells were immediately subjected to SERS and dark-field imaging using a Renishaw Raman system at different time points.

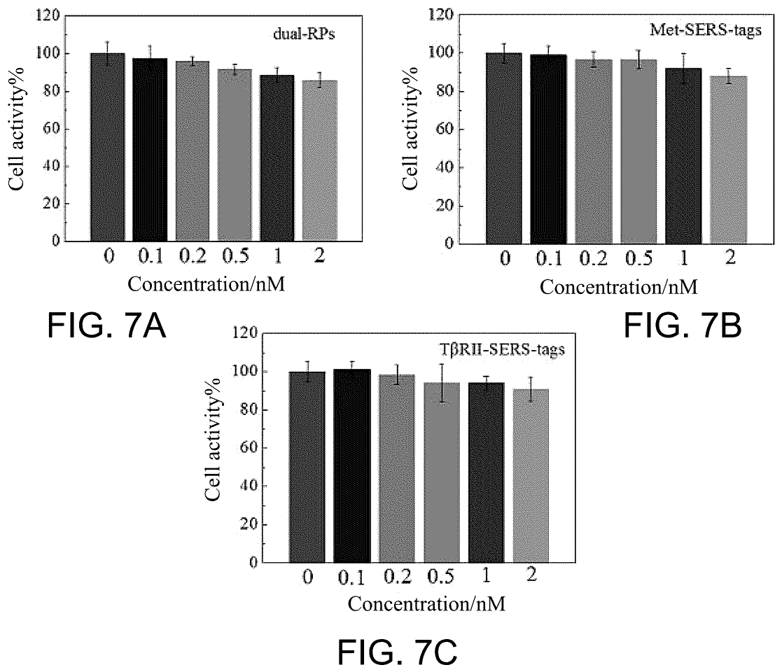

As shown in FIG. 7, the dual-RPs in Panel A, the Met-SERS tags in Panel B, and the TβRII-SERS tags in Panel C show good biocompatibility to the DU145 cells. Even when the concentration of AuNPs is as high as 2 nM, the cell activity is still higher than 85%.

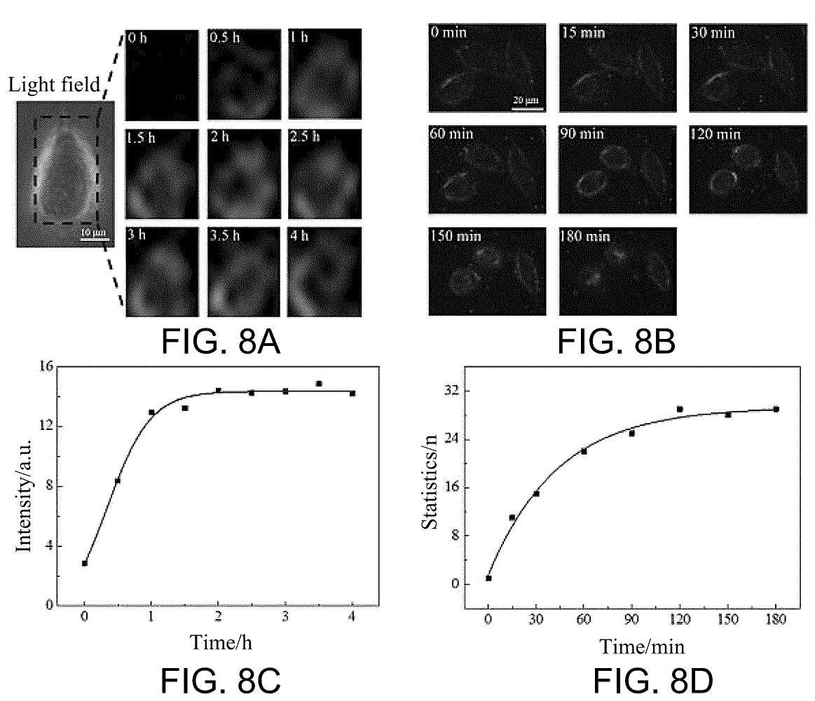

As shown in FIG. 8, Met is a cognate receptor of hepatocyte growth factor (HGF). HGF can activate downstream protein expression by inducing dimerization of Met. To sense the Met protein dimerization, the HGF-treated DU145 cells were first co-incubated with T-Met-1 and T-Met-2, into which then the dual-RPs and Met-SERS tags were added, followed by SERS imaging at intervals of 30 min. Panel A of FIG. 8 shows the time-dependent SERS visualization of the Met protein dimer, and Panel C of FIG. 8 is the corresponding time-dependent SERS signal intensity curve of the Met protein dimer. An obvious 4-MBA SERS signal appears after incubation for 30 min, and the signal reaches saturation after 2 h. At the same time, through dark-field microscopy imaging, it can be observed that a large number of 15Au-50Au network nanostructures are formed within 2 h, as shown in Panel B and Panel D of FIG. 8. Panel B is a dark-field microscopy imaging diagram, and Panel D is a statistical curve of the number of large particle bright spots assembled in Panel B, which is consistent with the results of SERS imaging.

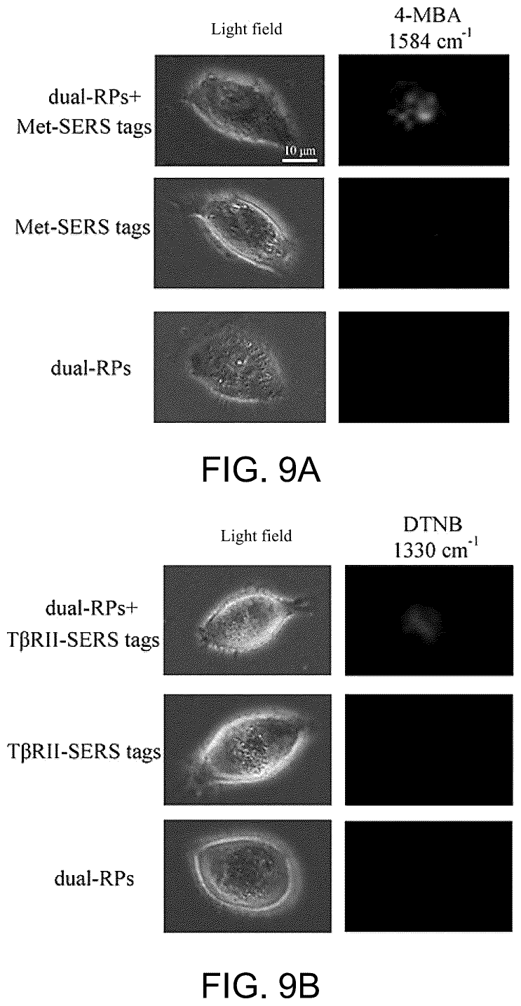

As shown in FIG. 9, for HGF-induced Met protein dimerization, almost no SERS signal of 4-MBA is collected in the DU145 cells co-cultured with the dual-RPs alone or the Met-SERS tags alone, as shown in Panel A of FIG. 9. However, when the DU145 cells were co-cultured with both the dual-RPs and the Met-SERS tags, an obvious 4-MBA SERS signal is detected due to the formation of 15Au-50Au network nanostructures assembled by the CHA reaction. Similarly, for TGFβ1-induced TβRII protein dimerization, as shown in Panel B of FIG. 9, an obvious SERS signal belonging to DTNB can be observed only when the DU145 cells are co-cultured with the dual-RPs and the TβRII-SERS tags, indicating that the proposed intercellular signaling visualization method can be applied to the imaging of Met and TβRII protein dimerization on the cell membrane.

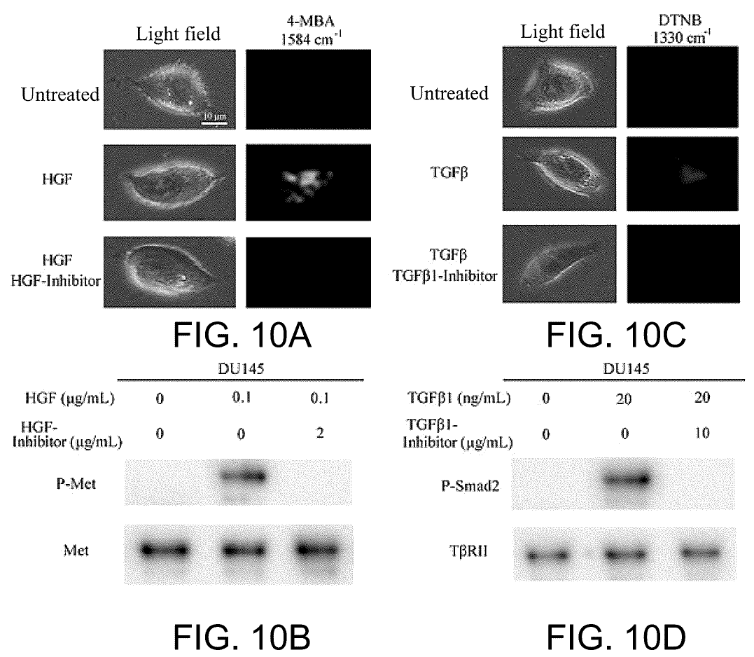

Panel A of FIG. 10 shows the SERS imaging of the DU145 cells before and after HGF treatment and the DU145 cells co-treated with HGF and an HGF inhibitor according to the present invention. In cells without HGF stimulation, almost no SERS signal of 4-MBA is observed, as shown in the untreated group in Panel A of FIG. 10, which indicates that the Met protein is mainly dispersed on the cell membrane in the form of monomers in the natural state. HGF-induced DU145 cells show an obvious 4-MBA SERS signal as shown in the HGF group in Panel A of FIG. 10, which is because the dimerization of the Met protein triggers the formation of network nanostructures. Once the dimerization of the Met protein is inhibited by an HGF inhibitor, such as that in the HGF-HGF-Inhibitor co-treatment group in Panel A of FIG. 10, it is difficult to detect the SERS signal of 4-MBA.

The example of the present invention further uses protein blotting methods to detect the phosphorylation level of Met under different treatment conditions, as shown in Panel B of FIG. 10. Compared with the untreated cells, the phosphorylation level of Met (phospho-Met, P-Met) in the HGF-treated cells is significantly increased. However, for cells pretreated with the HGF inhibitor, the expression of P-Met decreases. These results are consistent with the widely accepted hypothesis for the mechanism of the HGF/Met signaling pathway, that is, the formation of the dimer protein induces the autophosphorylation of the Met protein.

In addition, as shown in Panels C and D of FIG. 10, similar results are also obtained by studying the phosphorylation level of the corresponding Smad2 protein (i.e., TGFβ1/Smad2 signaling pathway) when TβRII protein dimerization is stimulated (TGFβ1) or inhibited (TGFβ1-Inhibitor). In summary, the proposed visualization method of intercellular signaling based on surface enhanced Raman spectroscopy can specifically and accurately image membrane protein dimerization on living cells.

### Example 5 Simultaneous SERS Imaging of Met Protein Dimerization and TβRII Protein Dimerization

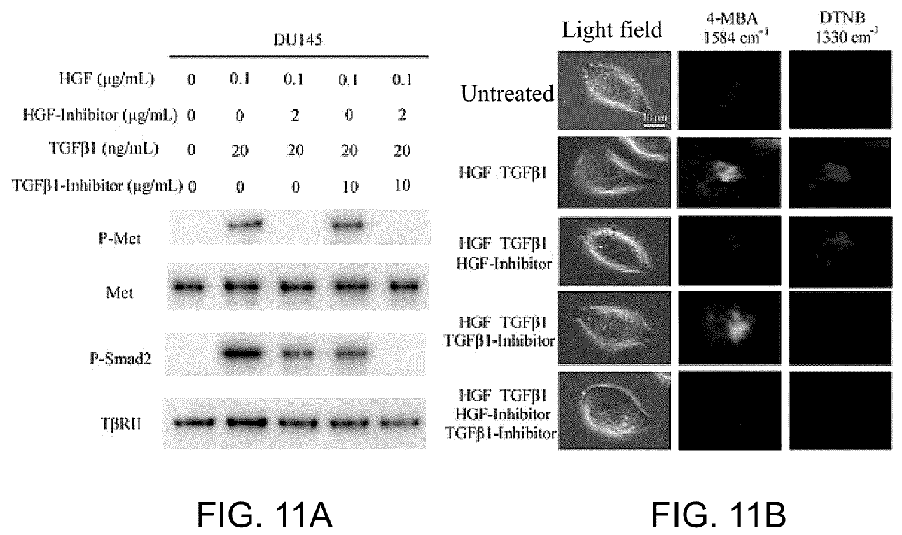

Studies have shown that the HGF and TGFβ1 signaling pathways have a complex network relationship. For example, TGFβ1 can promote the expression of an HGF receptor (Met); conversely, HGF can regulate (inhibit) the secretion of TGFβ1. The TGFβ1/Smad2 signaling pathway is generally characterized by the phosphorylation of the downstream Smad2 protein, while the HGF/Met signaling pathway can also lead to the phosphorylation of the Smad2 protein. Therefore, when both signaling pathways are present, traditional protein blotting methods cannot accurately determine which type of intercellular communication occurs. Panel A of FIG. 11 shows protein blotting methods of Met and TβRII protein dimerization on DU145 cells. Met and TβRII are expressed on DU145 cells regardless of special treatment. Significant upregulation of P-Met and P-Smad2 is observed in cells co-treated with HGF and TGFβ1, whereas no significant P-Met and P-Smad2 expression is found in cells pre-treated with HGF and TGFβ1 as well as the corresponding inhibitors. In the case of adding the HGF inhibitor without the TGFβ1 inhibitor, the HGF/Met signaling pathway is blocked and there is no obvious P-Met expression, while TGFβ1/Smad2 signaling pathway is still activated by TGFβ1 and the expression of P-Smad2 is upregulated. It is noteworthy that even if the TGF B1/Smad2 signaling pathway is blocked by the TGFβ1 inhibitor, namely, in the case of adding the TGFβ1 inhibitor without the HGF inhibitor, HGF can still induce Smad2 phosphorylation, and the expression of P-Met and P-Smad2 can be observed. Although the Smad protein mediates signals from receptor serine-threonine kinases (RSKs) of the TGFβ family, HGF, which signals through receptor tyrosine kinases (RTKs), can also mediate Smad-dependent gene activation and induce kinases downstream of the MAP kinase kinase 1 (MEK1) to rapidly phosphorylate endogenous Smad proteins.

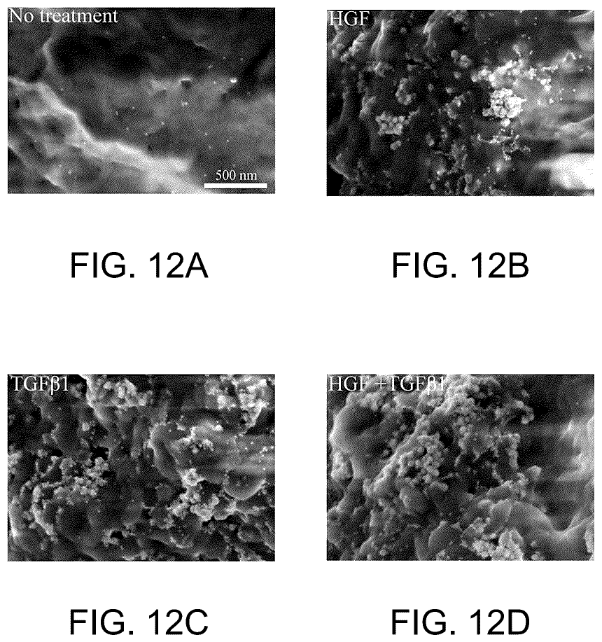

SERS imaging can provide a visual tool to identify specific signaling pathways. Panel B of FIG. 11 gives the SERS imaging of two cell communications based on membrane protein dimerization according to the present invention, i.e., the simultaneous dimerization of the Met protein and TβRII protein on DU145 cells. As shown in the HGF TGFβ1 group in Panel B of FIG. 11, for the DU145 cells co-treated with HGF and TGFB1, the Raman signals of 4-MBA and DTNB are significantly enhanced, while as shown in the untreated group in Panel B of FIG. 11, almost no SERS signal is detected in cells without induction treatment. Furthermore, both HGF- and TGFβ1-based signal transduction can be blocked by treatment with their corresponding inhibitors. As shown in the HGF TGFβ1 HGF-Inhibitor co-treatment group in Panel B of FIG. 11, a single HGF inhibitor can block the signaling of HGF. Similarly, as shown in the HGF TGFβ1 TGFβ1-Inhibitor co-treatment group in Panel B of FIG. 11, a single TGFβ1 inhibitor can block the signaling of TGFβ1. As shown in the HGF TGFβ1 HGF-Inhibitor TGFβ1-Inhibitor co-treatment group in Panel B of FIG. 11, both signaling pathways can be blocked by using both the HGF inhibitor and the TGFβ1 inhibitor. The SEM images shown in FIG. 12, wherein Panel A of FIG. 12 is the untreated blank group, strongly support the results of SERS imaging by comparing the particle assembly on the cell membrane surface treated by HGF alone as shown in Panel B of FIG. 12 or TGFβ1 alone as shown in Panel C of FIG. 12, or treated by HGF and TGFβ1 together as shown in Panel D of FIG. 12. Therefore, either the simultaneous dimerization of Met and TβRII proteins, or the individual dimerization of Met or TβRII proteins, can be accurately observed by the SERS method provided by the present invention without any mutual interference.

### Example 6 SERS Imaging of Cell Communication Between Cancer Cells and Stromal or Stem Cells

In this example, a Transwell system was used to carry out the cell co-culture test. The specific experimental process was as follows: DU145 cells were seeded in a 6-well plate and cultured in a DMEM medium containing fetal bovine serum FBS-10% for 24 h, and then starved in a DMEM medium without FBS for 24 h. At the same time, WPMY-1 cells or BMSCs were cultured in a chamber (semipermeable membrane pore size 0.4 μm) containing 0.5 mL of a DMEM medium without FBS at a density of 5.0×10⁴ cells/mL. After 48 h of co-culture, the DU145 cells were incubated with 100 nM T-Met-1, T-Met-2, T-TβRII-1, and T-TβRII-2 for 10 min and washed three times with PBS for imaging detection. The DU145 cells were immediately subjected to SERS imaging using a Renishaw Raman system after the addition of dual-RPs and SERS tags (including Met-SERS tags and TβRII-SERS tags).

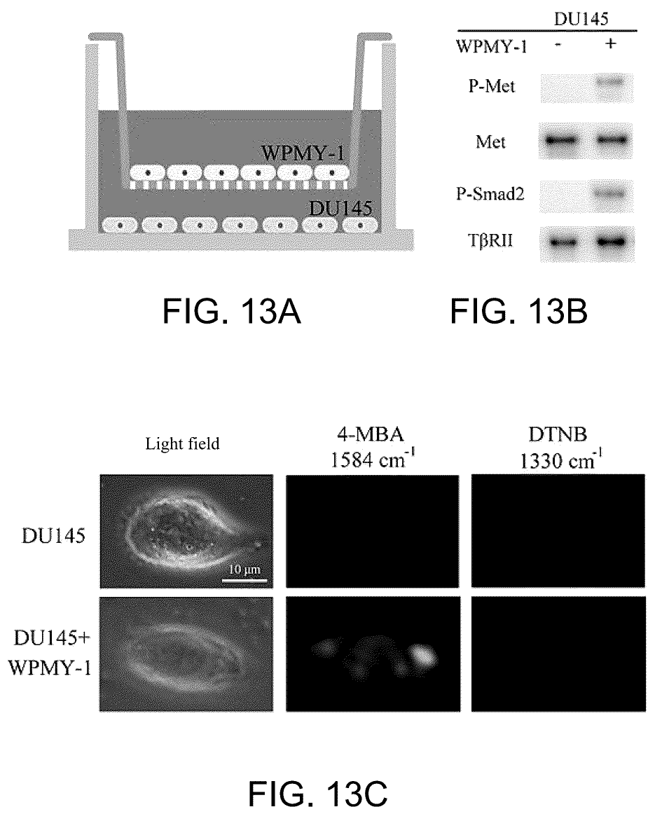

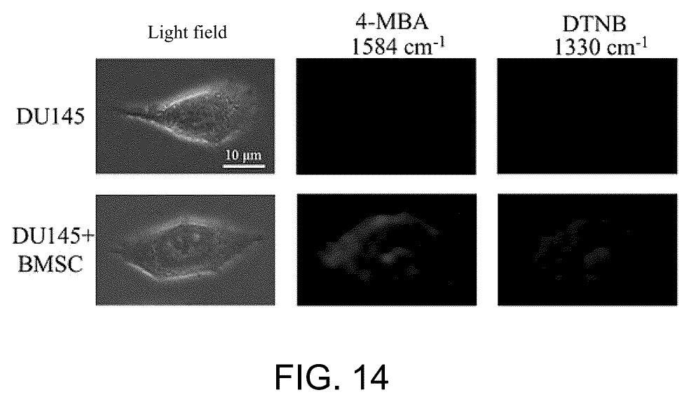

Determining epithelium-stroma interactions (i.e., tumor-stroma interactions) is of great importance during local infiltration of prostate cancer, because the stromal cells of the prostate produce many types of growth factors, which act in a paracrine manner on epithelial cells, including cancer cells. So far, no method for visualizing intercellular signaling based on SERS technology has been reported. Here, the present invention has studied the possible signal transmission pathway between DU145 and WPMY-1 cells by co-culturing the two types of cells in a Transwell system, as shown in Panel A of FIG. 13. The protein blotting method shown in Panel B of FIG. 13 demonstrates that the signal between DU145 and WPMY-1 cells results in phosphorylation of Met and Smad2 proteins in DU145 cells. However, this result cannot determine whether the two cells communicate through HGF alone or through cell communication mediated by a combination of HGF and TGFβ1. With the help of SERS imaging, only dimerization of the Met protein is observed compared with DU145 cells cultured alone, which clearly indicates that the communication between DU145 and WPMY-1 cells mainly depends on the HGF/Met signaling pathway, as shown in Panel C of FIG. 13. In addition, stem cells (such as BMSCs) can secrete a variety of cytokines, such as HGF and TGFβ1, to regulate various biological processes in organisms. However, visualization with high sensitivity of the complex intercellular signal transduction and intracellular signaling pathways in this process remains a challenge. The SERS technology is used to image dimers of cell membrane proteins induced by stem cell secretions to reveal the key role of stem cells in biological regulation. As shown in the SERS images in FIG. 14, signals of dimerization of Met and TβRII proteins can be observed from DU145 cells when co-cultured with BMSCs, indicating that BMSCs can simultaneously regulate the physiological state of the cells through the HGF/Met and TGFβ1/Smad2 signaling pathways.

1. An in situ detection kit for a membrane protein dimerization state, comprising:
a first membrane protein group anchoring chain, a recognition probe, and a first SERS probe;
the first membrane protein group anchoring chain comprises two first membrane protein anchoring chains P1-T1 and P1-T2 respectively for anchoring two receptor protein monomers on a first membrane protein, the P1-T1 and P1-T2 are capable of proximity hybridization during dimerization of the first membrane protein, the P1-T1 and P1-T2 each comprise an aptamer sequence, a free sequence, a hybridization sequence, and a trigger sequence sequentially, the aptamer sequences and the free sequences in the P1-T1 and P1-T2 are the same, the hybridization sequences in the P1-T1 and P1-T2 are completely complementary to each other and have only 6-8 bases; the aptamer sequences in the P1-T1 and P1-T2 bind to the two receptor protein monomers respectively, and when the two receptor protein monomers form a dimer under an action of a dimerization inducer, the P1-T1 and P1-T2 form a double-stranded T-shaped structure through complementation of the hybridization sequences;
the recognition probe is prepared by modifying large-particle AuNPs with a first RP hairpin, and the first RP hairpin is assembled from DNA sequences and is capable of specifically recognizing the trigger sequences in the first membrane protein anchoring chains to form a first complex with the double-stranded T-shaped structure, wherein the large-particle AuNPs have a diameter ranging from 40 to 70 nm; and
the first SERS probe is prepared by anchoring a first SERS hairpin and a first Raman molecule on small-particle AuNPs, and the first SERS hairpin is assembled from DNA sequences and has a sequence complementary to that of the first RP hairpin, wherein the small-particle AuNPs have a diameter ranging from 10 to 30 nm, and the first Raman molecule is any one of 4-MBA, DTNB or 4-ATP.

2. The in situ detection kit for the membrane protein dimerization state according to claim 1, further comprising a second membrane protein group anchoring chain for anchoring a second membrane protein, and the second membrane protein group anchoring chain also comprises two anchoring chains P2-T1 and P2-T2, respectively for anchoring two receptor protein monomers on the second membrane protein, the P2-T1 and P2-T2 are also capable of proximity hybridization during dimerization of the second membrane protein to form a double-stranded T-shaped structure, the P2-T1 and P2-T2 also each comprise four parts: an aptamer sequence, a free sequence, a hybridization sequence, and a trigger sequence, and the hybridization sequences are complementary to each other, and also have only 6-8 complementary bases;
the recognition probe is a dual recognition probe, the dual recognition probe is prepared by modifying the large-particle AuNPs with the first RP hairpin and a second RP hairpin simultaneously; the second RP hairpin is assembled from DNA sequences, and is capable of specifically recognizing the trigger sequences in the second membrane protein group anchoring chain to form a second complex; the DNA sequences of the first RP hairpin and the second RP hairpin cannot form a stable hybridization relationship with each other, and the first RP hairpin and the second RP hairpin respectively bind to the trigger sequences of the first membrane protein anchoring chains and the trigger sequences of the second membrane protein anchoring chains to open structures of hairpins; and
the in situ detection kit further comprises a second SERS probe, the second SERS probe is prepared by anchoring a second SERS hairpin and a second Raman molecule on the small-particle AuNPs, the second SERS hairpin is assembled from DNA sequences and has a sequence complementary to that of the second RP hairpin; the second Raman molecule is selected from any one of 4-MBA, DTNB or 4-ATP, but is different from the first Raman molecule.

3. The in situ detection kit for the membrane protein dimerization state according to claim 2, wherein the DNA sequences of the first RP hairpin and the second RP hairpin are shown in SEQ ID NO: 5 and SEQ ID NO: 7, respectively.

4. The in situ detection kit for the membrane protein dimerization state according to claim 2, wherein the DNA sequences of the first RP hairpin and the second RP hairpin are shown in SEQ ID NO: 13 and SEQ ID NO: 15, respectively.

5. The in situ detection kit for the membrane protein dimerization state according to claim 1, wherein the large-particle AuNPs have a diameter of 50 nm, and the small-particle AuNPs have a diameter of 15 nm.

6. The in situ detection kit for the membrane protein dimerization state according to claim 1, wherein the hybridization sequence has only 7 bases.

7. A use of the in situ detection kit for the membrane protein dimerization state according to claim 2 in simultaneous detection of an Met protein dimerization state and a TβRII protein dimerization state, wherein the recognition probe is a dual recognition probe for recognition of two membrane proteins Met and TβRII proteins, the first RP hairpin is H1-Met, and the second RP hairpin is H1-TβRII; the first SERS probe is an Met-SERS tag, wherein the first SERS hairpin is H2-Met; the second SERS probe is a TβRII-SERS tag, wherein the second SERS hairpin is H2-TβRII; and
the use comprises following steps: first, incubating cells to be detected with anchoring chains T-Met-1, T-Met-2, T-TβRII-1 and T-TβRII-2 in a medium, so that the anchoring chains bind to corresponding Met or TβRII proteins on a cell membrane; washing with PBS, and then incubating the cells with the dual recognition probe, the Met-SERS tag and the TβRII-SERS tag; washing the cells with PBS, and then performing SERS imaging of protein dimers on living cells.

8. The use according to claim 7, wherein the T-Met-1 has a sequence shown in SEQ ID NO: 1, the T-Met-2 has a sequence shown in SEQ ID NO: 2, the T-TβRII-1 has a sequence shown in SEQ ID NO: 3, the T-TβRII-2 has a sequence shown in SEQ ID NO: 4, the H1-Met has a sequence shown in SEQ ID NO: 5, the H2-Met has a sequence shown in SEQ ID NO: 6, the H1-TβRII has a sequence shown in SEQ ID NO: 7, and the H2-TβRII has a sequence shown in SEQ ID NO: 8.

9. The use according to claim 7, wherein the T-Met-1 has a sequence shown in SEQ ID NO: 9, the T-Met-2 has a sequence shown in SEQ ID NO: 10, the T-TβRII-1 has a sequence shown in SEQ ID NO: 11, the T-TβRII-2 has a sequence shown in SEQ ID NO: 12, the H1-Met has a sequence shown in SEQ ID NO: 13, the H2-Met has a sequence shown in SEQ ID NO: 14, the H1-TβRII has a sequence shown in SEQ ID NO: 15, and the H2-TβRII has a sequence shown in SEQ ID NO: 16.

10. The use according to claim 7, wherein a molar ratio of the dual recognition probe, the Met-SERS tag and the TβRII-SERS tag is 1:25:2.
